(19) United States
(12) Patent Application Publication
(10) Pub. No.: US 2019/0239738 A1
(43) Pub. Date: Aug. 8, 2019

(54) OPTICAL SCANNING ENDOSCOPE DEVICE

(71) Applicant: OLYMPUS CORPORATION, Tokyo (JP)

(72) Inventor: Takeshi MORI, Tokyo (JP)

(73) Assignee: OLYMPUS CORPORATION, Tokyo (JP)

(21) Appl. No.: 16/385,113

(22) Filed: Apr. 16, 2019

Related U.S. Application Data
(63) Continuation of application No. PCT/JP2016/083282, filed on Nov. 9, 2016.

(51) Int. Cl. A61B 1/07; A61B 1/00; A61B 1/06; G02B 23/24

(52) U.S. Cl. CPC A61B 1/07; A61B 1/00096; G02B 23/2469; A61B 1/00006; G02B 23/2423; A61B 1/0661

(57) ABSTRACT

An optical scanning endoscope device includes: a light source that emits illumination light; an optical fiber that emits the illumination light from a distal end; an optical scanner that scans the illumination light on a subject; a return-light detector that detects return light returning from the subject; an emitted-light-amount detector that detects an amount of a portion of the illumination light emitted from the distal end; a converter that converts the detected amount of light into an amount of entire illumination light emitted from the distal end; a controller that controls an amount of the illumination light emitted by the light source based on the converted amount of emitted light; and a calculator that calculates an actual amount of the illumination light based on the detected amount of light and the detected amount of return light. The converter converts the actual amount into the amount of entire emitted illumination light.

### CROSS-REFERENCE TO RELATED APPLICATIONS

This is a continuation of International Application PCT/JP2016/083282, with an international filing date of Nov. 9, 2016, which is hereby incorporated by reference herein in its entirety.

### TECHNICAL FIELD

The present invention relates to an optical scanning endoscope device.

### BACKGROUND ART

There is a known optical scanning endoscope device that scans illumination light on a subject by emitting illumination light towards the subject from a vibrating distal end of an optical fiber, and allowing return light from the subject to be observed (refer to, for example, Patent Literature 1). In order to detect the balance among the amounts of R, G, and B light in illumination light, the scanning endoscope in Patent Literature 1 is provided with an amount-of-light detection unit for white balance for receiving a portion of the illumination light emitted from the optical fiber.

### CITATION LIST

### Patent Literature

### {PTL 1}

PCT International Publication No. WO 2016/079768

### SUMMARY OF INVENTION

One aspect of the present invention is directed to an optical scanning endoscope device including: a light source configured to emit illumination light; an optical fiber configured to guide the illumination light emitted from the light source to emit the illumination light from a distal end towards a subject; an optical scanner configured to vibrate the distal end of the optical fiber in a radial direction of the optical fiber to scan the illumination light on the subject; a return-light detector configured to detect return light that returns from the subject irradiated with the illumination light; an emitted-light-amount detector configured to detect an amount of a portion of the illumination light emitted from the distal end of the optical fiber; a converter configured to convert the amount of light detected by the emitted-light-amount detector into an amount of entire illumination light emitted from the distal end of the optical fiber; a controller configured to control an amount of the illumination light emitted by the light source on a basis of the amount of emitted light converted by the converter; and a calculator configured to calculate an actual amount of the illumination light detected by the emitted-light-amount detector on a basis of the amount of light detected by the emitted-light-amount detector and the amount of return light detected by the return-light detector, wherein the converter is configured to convert, into the amount of entire emitted illumination light, the actual amount of light calculated by the calculator.

Another aspect of the present invention is directed to an optical scanning endoscope device including: a light source configured to emit illumination light; an optical fiber configured to guide the illumination light emitted from the light source to emit the illumination light from a distal end towards a subject; an optical scanner configured to vibrate the distal end of the optical fiber in a radial direction of the optical fiber to scan the illumination light on the subject; a return-light detector configured to detect return light that returns from the subject irradiated with the illumination light; an emitted-light-amount detector configured to detect an amount of a portion of the illumination light emitted from the distal end of the optical fiber; a converter configured to convert the amount of light detected by the emitted-light-amount detector into an amount of entire illumination light emitted from the distal end of the optical fiber; and a controller configured to control the amount of the illumination light emitted by the light source on a basis of the amount of emitted light converted by the converter, wherein the controller is configured to control the amount of the illumination light emitted by the light source on a basis of the amount of emitted light converted from the amount of light that is detected by the emitted-light-amount detector when the distal end of the optical fiber is stationary or when an amplitude of vibration of the distal end is smaller than a prescribed threshold value.

### BRIEF DESCRIPTION OF DRAWINGS

FIG. 1 is a diagram showing the entire configuration of an optical scanning endoscope device according to a first embodiment of the present invention.

FIG. 2 is a longitudinal sectional view showing the internal configuration of a distal end portion of a scope of the optical scanning endoscope device in FIG. 1.

FIG. 3 is a diagram showing the entire configuration of an optical scanning endoscope device according to a second embodiment of the present invention.

FIG. 4 is a diagram showing the entire configuration of an optical scanning endoscope device according to a third embodiment of the present invention.

FIG. 5 is a longitudinal sectional view of a distal end portion of the scope, showing a modification of a return-light detection unit.

FIG. 6 is a longitudinal sectional view of a distal end portion of the scope, showing a modification of an emitted-light PD.

### DESCRIPTION OF EMBODIMENTS

### First Embodiment

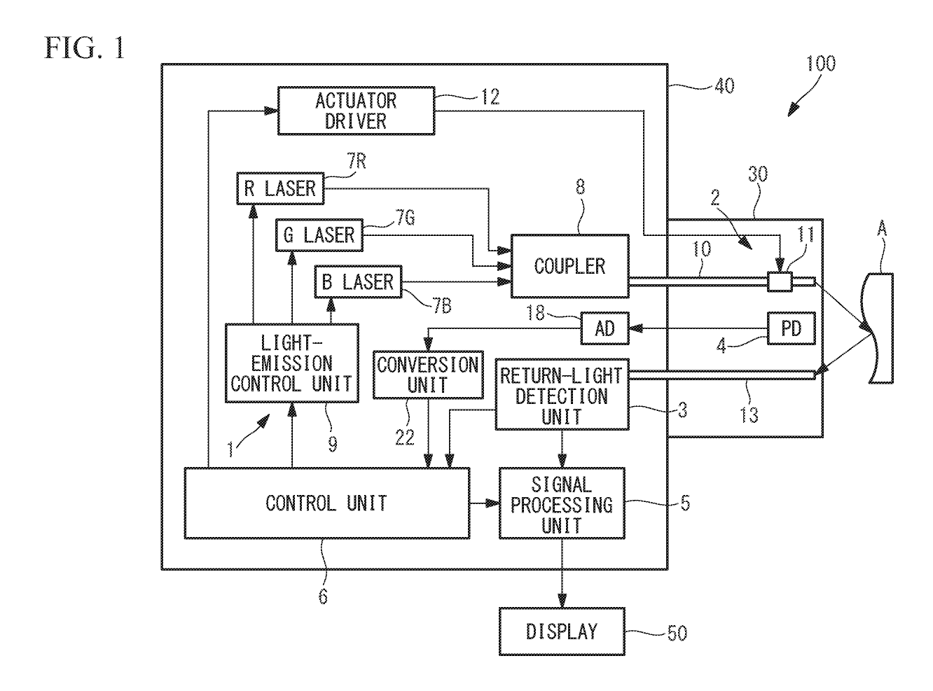

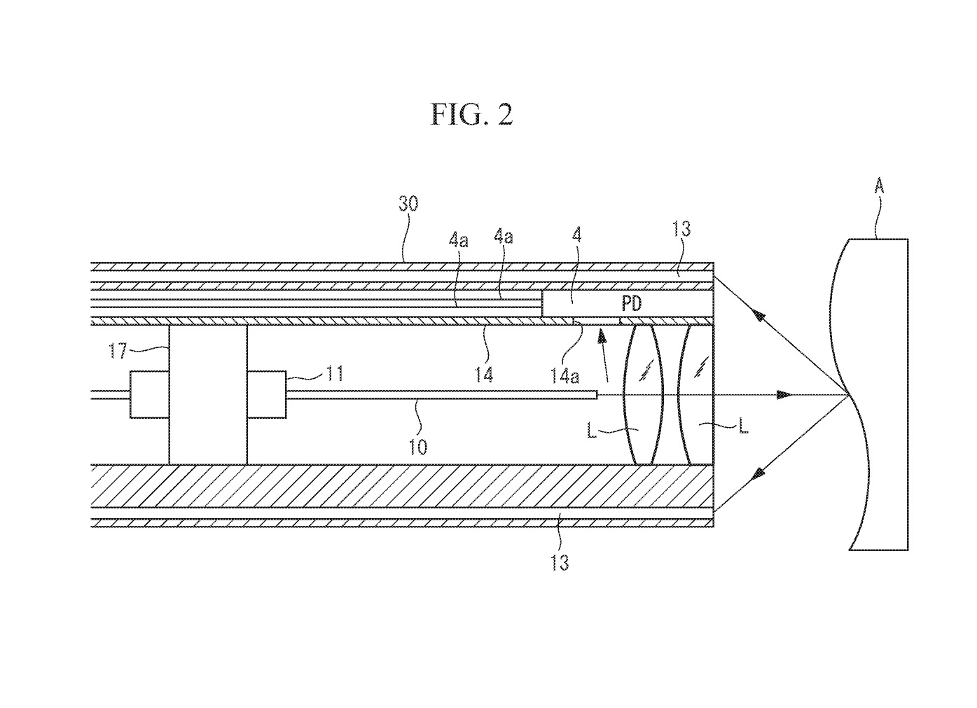

An optical scanning endoscope device 100 according to a first embodiment of the present invention will now be described with reference to FIGS. 1 and 2.

As shown in FIG. 1, the optical scanning endoscope device 100 according to this embodiment includes: an elongated scope 30 that is inserted into the body; a control device main body 40 connected to the basal end of the scope 30; and a display 50 connected to the control device main body 40.

In addition, the optical scanning endoscope device 100 includes: a light source unit 1 for outputting illumination light; an illumination fiber (optical fiber) 10 that guides the illumination light from the light source unit 1 and that emits the illumination light towards a subject A; an optical scanning section 2 for scanning the illumination light on the subject A; a return-light detection unit 3 for detecting return light from the subject A; an emitted-light photodiode (emitted-light-amount detection unit) 4 for detecting the amount of a portion of illumination light emitted from the illumination fiber 10 of the optical scanning section 2; a conversion unit 22 for converting the amount of light detected by the emitted-light photodiode (PD) 4 into the amount of entire emitted illumination light; a signal processing unit 5 for forming image data of the subject A on the basis of the intensity of return light and the irradiation position of illumination light; and a control unit 6 for controlling the entire optical scanning endoscope device 100.

The light source unit 1 is provided in the control device main body 40. The light source unit 1 includes: three laser light sources (light sources) 7R, 7G, and 7B for emitting red (R), green (G), and blue (B) laser beams, respectively; a coupler 8 for coaxially combining the R, G, and B laser beams emitted from the laser light sources 7R, 7G, and 7B; and a light-emission control unit 9 for controlling the laser light sources 7R, 7G, and 7B.

The laser light sources 7R, 7G, and 7B are, for example, DPSS lasers (diode pumped solid state lasers) or laser diodes.

The light-emission control unit 9 causes the laser light sources 7R, 7G, and 7B to sequentially emit pulsed light at certain time intervals according to a control signal from the control unit 6, thus generating R, G and B laser beams, in order, as illumination light.

The illumination fiber 10 is a single-mode optical fiber. As shown in FIG. 2, the illumination fiber 10 is disposed in the scope 30 along the longitudinal direction, and the basal end of the illumination fiber 10 is connected to the coupler 8. The illumination fiber 10 guides illumination light supplied from the coupler 8 and emits the illumination light from the distal end towards the subject A facing the distal end surface of the scope 30. Reference sign L denotes lenses for focusing the illumination light emitted from the illumination fiber 10.

The optical scanning section 2 includes: an actuator 11 provided on the illumination fiber 10; and an actuator driver 12 provided in the control device main body 40.

The actuator 11 is, for example, a piezoelectric actuator provided with a piezoelectric element and is attached to the illumination fiber 10 at a position separated from the distal end of the illumination fiber 10 towards the basal end thereof. Reference sign 17 denotes a fixing part for fixing a longitudinal-direction intermediate position of the illumination fiber 10 to the frame of the scope 30 so as to support the distal end portion of the illumination fiber 10 in a cantilever form. The actuator 11 vibrates the distal end of the illumination fiber 10 in a direction intersecting the longitudinal direction of the illumination fiber 10 as a result of an AC voltage being applied thereto from the actuator driver 12. By doing so, illumination light emitted from the distal end of the illumination fiber 10 is scanned.

The trajectory of vibration of the distal end of the illumination fiber 10 (i.e., scanning trajectory of illumination light) is controlled according to the amplitude and the phase of the AC voltage. For example, the control unit 6 controls the actuator driver 12 so that the actuator driver 12 generates an AC voltage that causes illumination light to be scanned along a spiral scanning trajectory and applies that AC voltage to the actuator 11.

The return-light detection unit 3 includes: a photodetector (not shown in the figure) for detecting return light via a light-receiving fiber 13 that is disposed in the scope 30 along the longitudinal direction; and an AD converter (not shown in the figure) for digitizing an electrical signal corresponding to the amount of return light detected by the photodetector.

The light-receiving fiber 13 is a multimode optical fiber. The return light that has returned from the subject A to the distal end of the scope 30 is received by the light-receiving fiber 13 and is guided to the return-light detection unit 3 by the light-receiving fiber 13. In order to increase the amount of received return light, the return-light detection unit 3 may be configured to receive return light from a plurality of the light-receiving fibers 13 arranged in the circumferential direction of the scope 30.

The return-light detection unit 3 transmits, to the signal processing unit 5, the value of the amount of return light obtained by the AD converter. The return-light detection unit 3 may further apply processing, such as offset correction and amplification, to the electrical signal of the return light.

Note that the laser light sources 7R, 7G, and 7B may output a series of R, G, and B laser beams, respectively, so that the coupler 8 may combine the R, G, and B laser beams to supply a white laser beam as illumination light to the illumination fiber 10. In this case, a color separation element (not shown in the figure) for decomposing white return light received by the light-receiving fiber 13 into R, G, and B wavelength components, as well as three photodetectors for detecting the R, G, and B wavelength components, respectively, decomposed by the color separation element, are provided.

A light-blocking wall 14 for shutting off illumination light is provided on the outward side in the radial direction of the illumination fiber 10, and the emitted-light PD 4 is provided on a side more outward than the light-blocking wall 14 in the radial direction. The light-blocking wall 14 has a light-transmitting window 14a at a position facing the distal end of the illumination fiber 10 in the radial direction, and only light passing through the window 14a is incident on the emitted-light PD 4. By doing so, the amount of a portion of illumination light emitted from the distal end of the illumination fiber 10 is detected by the emitted-light PD 4. Reference sign 4a denotes wiring lines for the emitted-light PD 4.

The amount of illumination light detected by the emitted-light PD 4 correlates with the amount of entire illumination light emitted from the distal end of the illumination fiber 10. Therefore, the amount of entire emitted illumination light can be estimated from the amount of light detected by the emitted-light PD 4.

The emitted-light PD 4 transmits, to the conversion unit 22 via an AD converter 18, an electrical signal corresponding to the amount of detected light. An amplifier for amplifying the electrical signal may be provided between the emitted-light PD 4 and the AD converter 18.

The conversion unit 22 converts the amount of light received from the emitted-light PD 4 into the amount of entire emitted illumination light (amount of entire emitted light) exiting from the distal end of the illumination fiber 10 and transmits the converted amount of entire emitted light to the control unit 6.

The signal processing unit 5 forms image data by associating the value of the amount of return light received from the return-light detection unit 3 with the irradiation position (described later) of illumination light received from the control unit 6. The image data which has been formed is transmitted from the signal processing unit 5 to the display 50 and is displayed on the display 50. The signal processing unit 5 may transmit the image data to the display 50 after applying arbitrary image processing (e.g., level correction, interpolation processing, enhancement processing, γ processing, etc.) to the image data.

As described above, the control unit 6 controls the light-emission timings of the laser light sources 7R, 7G, and 7B via the light-emission control unit 9. In addition, the control unit 6 calculates the irradiation position of illumination light from a control signal transmitted to the actuator driver 12 and transmits information on the calculated irradiation position to the signal processing unit 5.

In addition, the control unit 6 adjusts the amount of light emitted from the laser light sources 7R, 7G, and 7B by transmitting, to the light-emission control unit 9, a control signal for bringing the amount of entire emitted illumination light that has been received from the conversion unit 22 close to a target amount. More specifically, the control unit 6 controls the light-emission control unit 9 so as to increase the amount of light emitted from the laser light sources 7R, 7G, and 7B when the amount of entire emitted light is smaller than the target amount and so as to decrease the amount of light emitted from the laser light sources 7R, 7G, and 7B when the amount of entire emitted light is larger than the target amount.

The signal processing unit 5 and the control unit 6 may be realized, for example, by a computer including: a CPU (central processing unit); and a storage device for storing programs that make the CPU execute the above-described processing of the signal processing unit 5, the conversion unit 22, and the control unit 6.

Next, the operation of the optical scanning endoscope device 100 with this configuration will be described.

When the control unit 6 starts transmission of a control signal to the light-emission control unit 9 and the actuator driver 12, illumination light is emitted from the vibrating distal end of the illumination fiber 10. The emitted illumination light is radiated on the subject A facing the distal end surface of the scope 30 and is scanned on the subject A.

A portion of illumination light emitted from the distal end of the illumination fiber 10 is detected by the emitted-light PD 4 via the window 14a in the light-blocking wall 14. The conversion unit 22 obtains the amount of entire illumination light emitted from the distal end of the illumination fiber 10 on the basis of the amount of light received from the emitted-light PD 4. The control unit 6 feedback-controls the amounts of the laser beams emitted by the laser light sources 7R, 7G, and 7B so that the amount of entire emitted light coincides with the target amount. By doing so, the amount of illumination light that is emitted from the illumination fiber 10 and that is radiated on the subject A is controlled so as to coincide with the target amount.

Return light of illumination light reflected at the subject A is received by the light-receiving fiber 13 at the distal end surface of the scope 30 and is guided to the return-light detection unit 3. Then, the value of the amount of return light, serving as the value of each pixel of an image, is obtained in the return-light detection unit 3. When the obtained value of the amount of return light is associated with the irradiation position of illumination light in the signal processing unit 5, image data of the subject A is generated. The generated image data is displayed on the display 50.

In this manner, according to this embodiment, information on the amount of entire illumination light actually emitted from the distal end of the illumination fiber 10 is obtained from the emitted-light PD 4 provided in the vicinity of the distal end of the illumination fiber 10. In particular, by providing, between the illumination fiber 10 and the emitted-light PD 4, the light-blocking wall 14 that allows only the window 14a to transmit illumination light, it is possible to prevent return light entering the scope 30 via the lenses L, as well as illumination light diffusing in the scope 30, from entering the emitted-light PD 4, thereby allowing the emitted-light PD 4 to obtain information on a more accurate amount of emitted illumination light. This affords an advantage in that the amount of illumination light emitted from the illumination fiber 10 can be controlled to the target amount, irrespective of a difference between the amount of light emitted from the laser light sources 7R, 7G, and 7B and the amount of illumination light emitted from the illumination fiber 10.

In this embodiment, if the amount of light detected by the emitted-light PD 4 is so small that the amount of entire emitted light converted by the conversion unit 22 is less than a prescribed threshold value even though the laser light sources 7R, 7G, and 7B are emitting light, the control unit 6 may control the light-emission control unit 9 so as to stop light emission from the laser light sources 7R, 7G, and 7B.

By doing so, on the basis of the amount of light detected by the emitted-light PD 4, it is possible to detect that illumination light is not normally guided due to an abnormality taking place in the optical path of the illumination light and to automatically stop light emission from the laser light sources 7R, 7G, and 7B. Instead of, or in addition to, stopping light emission from the laser light sources 7R, 7G, and 7B, an operator may be informed by means of display on the display 50, sound, or the like that illumination light is not normally guided.

In this embodiment, if the value of the amount of return light received from the return-light detection unit 3 is less than a prescribed threshold value for a certain time period, the control unit 6 may control the light-emission control unit 9 so as to decrease the amount of light emitted from the laser light sources 7R, 7G, and 7B.

For example, in the case where the subject A is not present near the distal end of the scope 30, the amount of return light detected by the return-light detection unit 3 continues to be less than a prescribed first threshold value because no return light is generated. In such a case, it is possible to automatically stop emission of illumination light to prevent illumination light from being emitted wastefully.

Alternatively, in the case where the subject A is present near the distal end of the scope 30, the control unit 6 may determine that the amount of illumination light radiated on the subject A is insufficient when the amount of return light is less than a prescribed second threshold value that is larger than the prescribed first threshold value, thereby controlling the light-emission control unit 9 so as to increase the amount of light emitted from the laser light sources 7R, 7G, and 7B.

By doing so, when an image is dark, the amount of illumination light can be automatically adjusted so that the image brightness is increased.

In addition, in this embodiment, the signal processing unit 5 may adjust the white balance of an image on the basis of the amount of R, G and B light acquired by the emitted-light PD 4.

### Second Embodiment

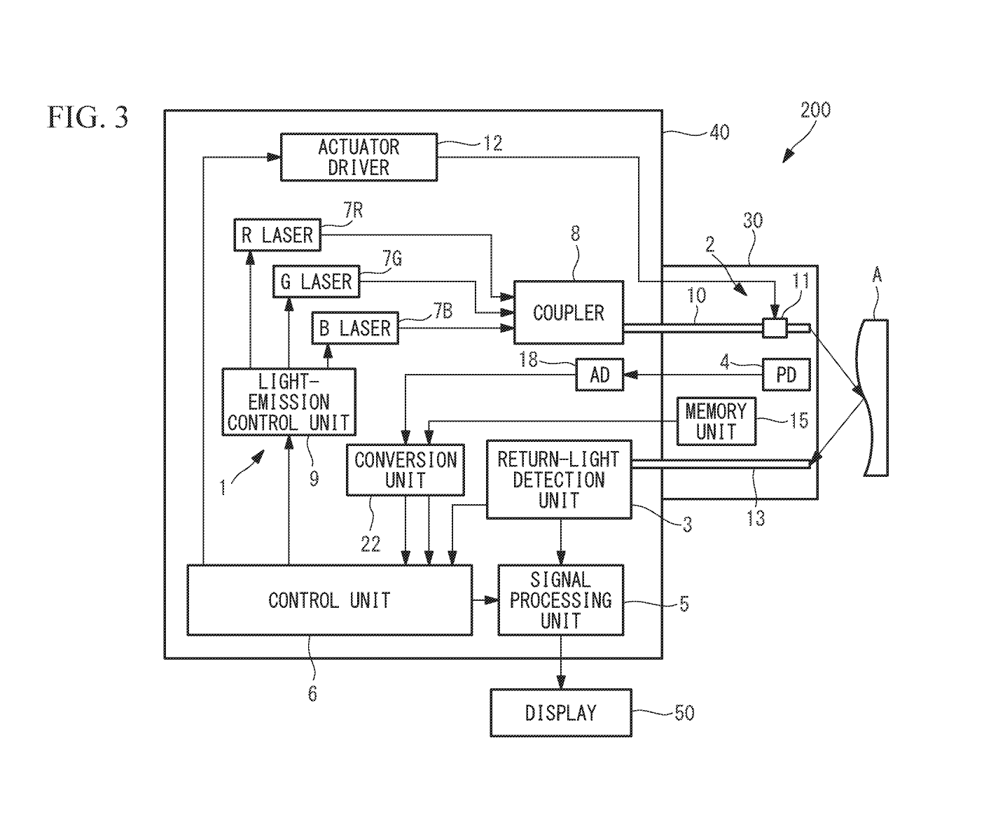

Next, an optical scanning endoscope device 200 according to a second embodiment of the present invention will be described with reference to FIG. 3.

In this embodiment, structures in common with those in the first embodiment will be denoted with the same reference signs, and descriptions thereof will be omitted.

In the optical scanning endoscope device 200 according to this embodiment, the scope 30 is attachable to and detachable from the control device main body 40, thereby making it possible to replace the scope 30 to be connected to the control device main body 40. In addition, as shown in FIG. 3, the scope 30 further includes a memory unit 15.

When the scope 30 is attached and detached, each of the optical fibers 10 and 13 and the wiring lines that span the scope 30 and the control device main body 40 can be connected and disconnected by means of a connector (not shown in the figure) at a longitudinal-direction intermediate position.

The memory unit 15 stores a correlative relationship between the amount of illumination light detected by the emitted-light PD 4 and the irradiation amount of illumination light emitted from the scope 30. The irradiation amount of illumination light is detected by a light-detecting device (not shown in the figure) disposed outside the scope 30. The correlative relationship is acquired by causing the emitted-light PD 4 and the light-detecting device to simultaneously detect illumination light while changing the amount of illumination light supplied from the light source unit 1 to the illumination fiber 10, at the time of manufacturing the scope 30. At this time, in order to prevent the detected irradiation amount of light from involving the amount of light based on return light, the light-detecting device detects illumination light under a condition where no return light is generated from the subject (e.g., a condition under which no object reflecting illumination light is present in front of the distal end surface of the scope 30).

The conversion unit 22 reads out the correlative relationship from the memory unit 15 provided in the scope 30 connected to the control device main body 40 and converts the amount of light received from the emitted-light PD 4 into the irradiation amount of illumination light on the basis of the correlative relationship.

The control unit 6 adjusts the amount of light emitted from the laser light sources 7R, 7G, and 7B by transmitting, to the light-emission control unit 9, a control signal for bringing the converted irradiation amount of light close to the target amount.

A portion of illumination light emitted from the distal end of the illumination fiber 10 is, for example, reflected at the lenses L and is not emitted from the distal end surface of the scope 30. Therefore, a difference arises between the amount of illumination light emitted from the illumination fiber 10 and the irradiation amount of illumination light that is emitted from the scope 30 and radiated on the subject. Furthermore, the difference between the amount of emitted light and the irradiation amount of light varies depending on manufacturing variations, such as the coating states and the tilting of the lenses L, and thus differs depending on the scope 30.

According to this embodiment, the laser light sources 7R, 7G, and 7B are controlled on the basis of the irradiation amount of illumination light from the scope 30, instead of the amount of illumination light emitted from the illumination fiber 10. This affords an advantage in that the irradiation amount of illumination light actually radiated on the subject A can be controlled to a desired amount. Furthermore, the irradiation amount of light not involving the amount of light based on return light can be detected by detecting illumination light under a condition where no return light is produced at the time of acquiring the correlative relationship, making it possible to acquire a more accurate correlative relationship. This affords an advantage in that even more accurate control is possible on the basis of such a correlative relationship so that the irradiation amount of illumination light radiated on the subject A exhibits a desired amount.

### Third Embodiment

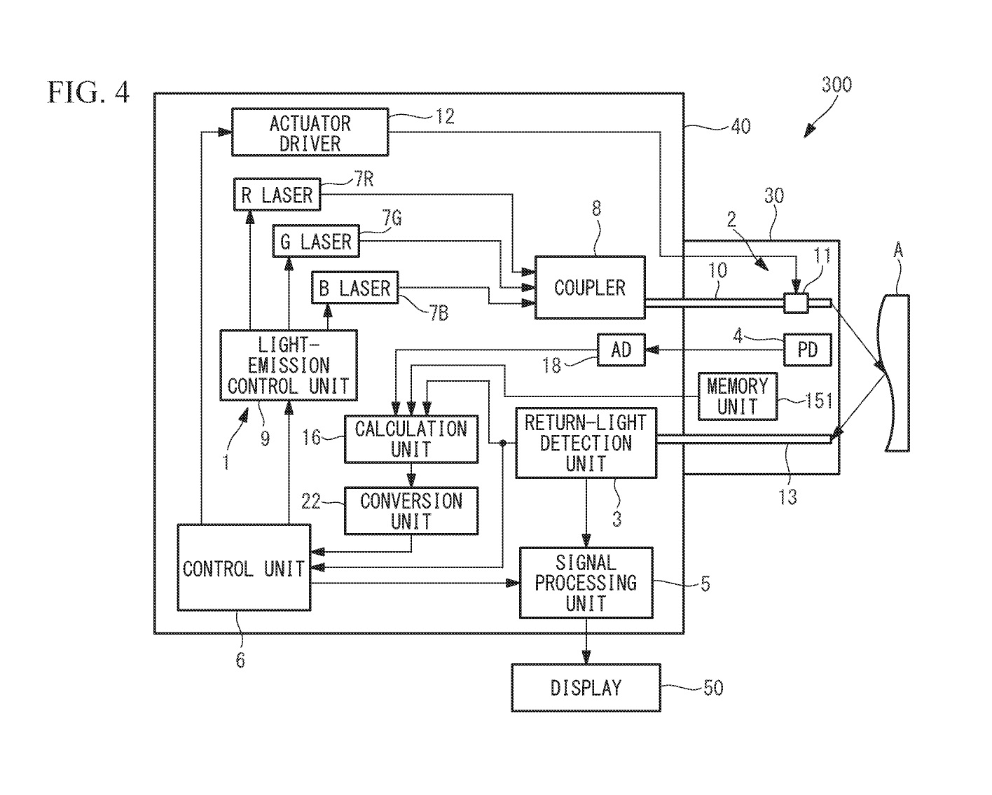

Next, an optical scanning endoscope device 300 according to a third embodiment of the present invention will be described with reference to FIG. 4.

In this embodiment, structures in common with those in the first and second embodiments will be denoted with the same reference signs, and descriptions thereof will be omitted.

In the optical scanning endoscope device 300 according to this embodiment, the scope 30 is attachable to and detachable from the control device main body 40, thereby making it possible to replace the scope 30 connected to the control device main body 40. In addition, as shown in FIG. 4, the scope 30 further includes a memory unit 151, and the control device main body 40 further includes a calculation unit 16.

When the scope 30 is attached and detached, each of the optical fibers 10 and 13 and the wiring lines that span the scope 30 and the control device main body 40 can be connected and disconnected by means of a connector (not shown in the figure) at a longitudinal-direction intermediate position.

The calculation unit 16 calculates an actual amount of illumination light, SIt, from expression (1) below on the basis of an amount of illumination light, SI, detected by the emitted-light PD 4 and an amount of return light, SR, detected by the return-light detection unit 3. The coefficient k is a value corresponding to the ratio of the amount of return light incident on the emitted-light PD 4 with respect to the amount of return light, SR, detected by the return-light detection unit 3.

SIt=SI−k×SR (1)

More specifically, the calculation unit 16 calculates the net amount of illumination light included in the light incident on the emitted-light PD 4 by subtracting the amount of light based on return light from the amount of light detected by the emitted-light PD 4.

The memory unit 151 stores the above-described coefficient k. The coefficient k is a value determined by detecting the amounts of light SIt, SI, and SR when the same amount of illumination light is supplied to the illumination fiber 10 at the time of manufacturing the scope 30. More specifically, the actual amount of light, SIt, is acquired by detecting illumination light emitted from the illumination fiber 10 by means of the emitted-light PD 4 under a condition where no return light is produced (condition where return light from the subject A is not guided to the scope 30). The amount of light, SI, is acquired by detecting illumination light emitted from the illumination fiber 10 by means of the emitted-light PD 4 under a condition where return light is produced (condition where the subject A is present). The amount of light, SR, is acquired by emitting illumination light from the illumination fiber 10 under a condition where return light is produced and then by detecting return light by means of the return-light detection unit 3.

The calculation unit 16 reads out the coefficient k from the memory unit 151 in the scope 30 connected to the control device main body 40 and calculates the actual amount of light, SIt, from expression (1) on the basis of the read-out coefficient k and the amounts of light SI and SR received from the emitted-light PD 4 and the return-light detection unit 3.

The conversion unit 22 converts the actual amount of light, SIt, calculated by the calculation unit 16, instead of the amount of light received from the emitted-light PD 4, into the amount of entire emitted illumination light. The subsequent control performed by the control unit 6 is the same as in the first embodiment.

According to this embodiment, the actual amount of illumination light incident upon the emitted-light PD 4 is calculated by removing the amount of light based on return light from the amount of light by means of the emitted-light PD 4, and the amount of light emitted from the laser light sources 7R, 7G, and 7B are controlled on the basis of the actual amount of light. This affords an advantage in that even more accurate control is possible so that the amount of illumination light emitted from the illumination fiber 10 exhibits a desired amount.

In the above-described first to third embodiments, the control unit 6 preferably controls the laser light sources 7R, 7G, and 7B on the basis of the amount of entire emitted light or the irradiation amount of light that has been converted from the amount of illumination light that is detected by the emitted-light PD 4 when the distal end of the illumination fiber 10 is stationary or when the amplitude of vibration of the distal end is smaller than a prescribed threshold value.

Because the distance between the distal end of the illumination fiber 10 and the emitted-light PD 4 changes greatly when the amplitude of vibration of the distal end of the illumination fiber 10 is large, the amount of illumination light incident upon the emitted-light PD 4 also changes. For this reason, it is difficult to detect an accurate amount of illumination light. Because the amount of illumination light incident on the emitted-light PD 4 becomes stable when the distal end of the illumination fiber 10 is stationary or when the amplitude of vibration of the distal end is small, it is possible to detect a more accurate amount of illumination light. Therefore, the amount of light emitted from the laser light sources 7R, 7G, and 7B can be controlled even more accurately on the basis of the amount of light when the distal end of the illumination fiber 10 is stationary or when the amplitude of vibration of the distal end is small.

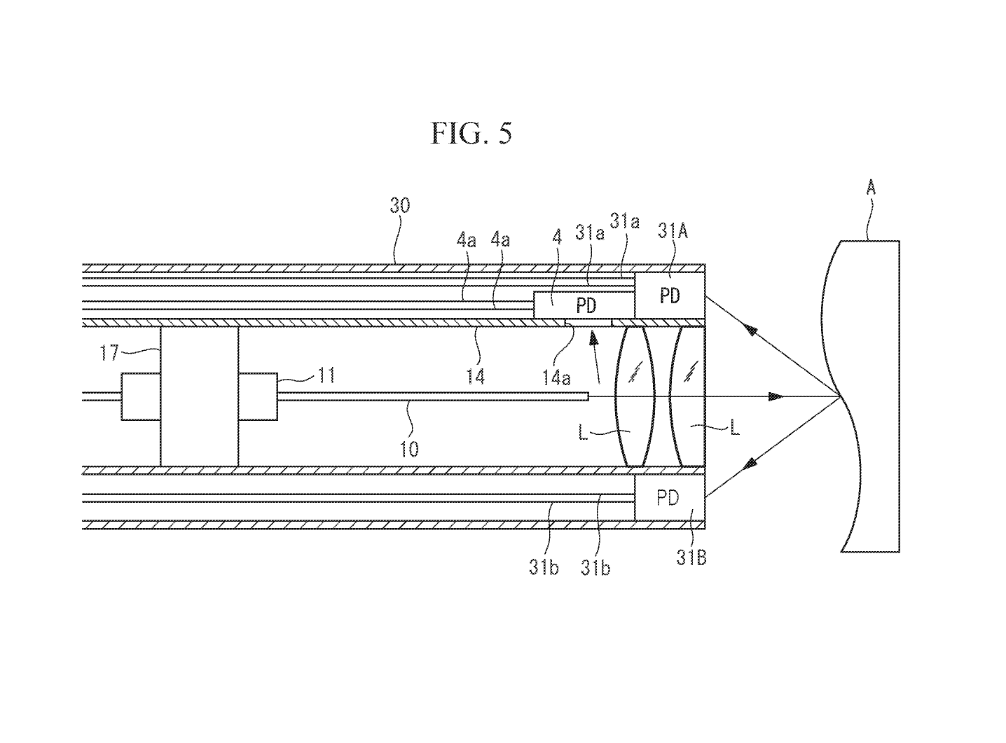

In the above-described first to third embodiments, return light is guided by the light-receiving fiber 13 from the distal end of the scope 30 to the control device main body 40, and the return light is detected by the return-light detection unit 3 provided in the control device main body 40. Instead of this, as shown in FIG. 5, return light may be detected by means of return-light photodiodes (return-light detection units) 31A and 31B provided at the distal end of the scope 30 in a manner arranged in the circumferential direction. In this case, an AD converter for analog-converting electrical signals output from the return-light PDs 31A and 31B is provided between the return-light photodiodes (PDs) 31A and 31B and the signal processing unit 5. Reference signs 31a and 31b denote wiring lines for the return-light PDs 31A and 31B, respectively.

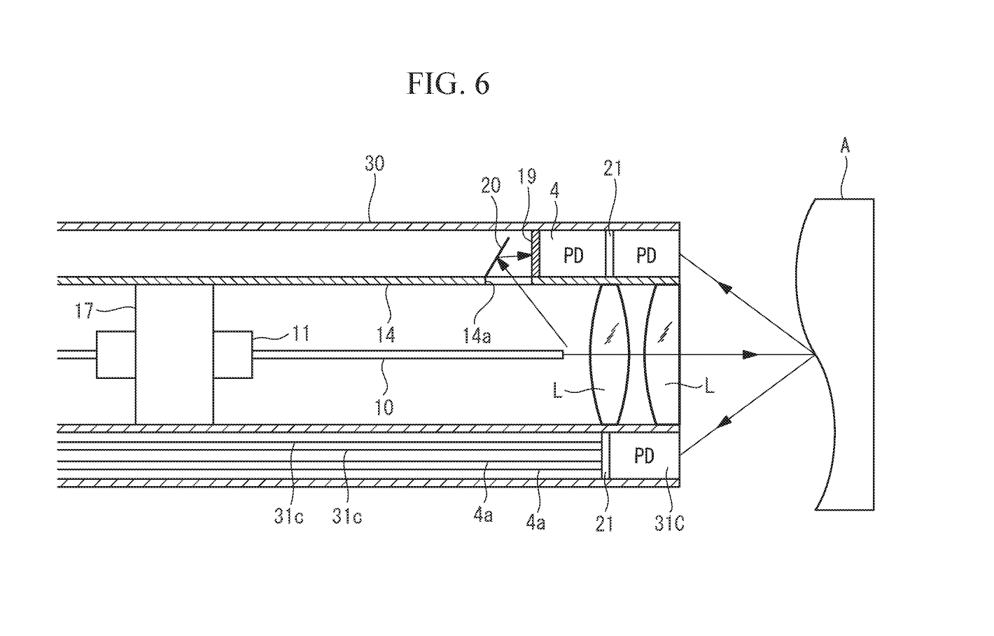

As shown in FIG. 6, in the above-described first to third embodiments, R, G, and B color filters 19 may be arranged on the incidence plane of the emitted-light PD 4 so that R, G, and B laser beams can be detected individually. Reference sign 20 denotes a reflecting member for reflecting a laser beam, reference sign 21 denotes substrates, and reference sign 31c denotes wiring lines for a return-light PD 31C. Although FIG. 6 shows a ring-shaped single return-light PD 31C, a plurality of the return-light PDs 31A and 31B arranged in the circumferential direction may be employed, as shown in FIG. 5.

By doing so, the amounts of light emitted from the laser light sources 7R, 7G, and 7B can be controlled individually on the basis of the amount of light of each color detected by the emitted-light PD 4.

As a result, the above-described embodiment leads to the following aspect.

One aspect of the present invention is directed to an optical scanning endoscope device including: a light source for emitting illumination light; an optical fiber that guides the illumination light emitted from the light source and that emits the illumination light from a distal end towards a subject; an optical scanning section that vibrates the distal end of the optical fiber in a radial direction of the optical fiber and that scans the illumination light on the subject; a return-light detection unit for detecting return light that returns from the subject irradiated with the illumination light; an emitted-light-amount detection unit for detecting the amount of a portion of the illumination light emitted from the distal end of the optical fiber; a conversion unit for converting the amount of light detected by the emitted-light-amount detection unit into the amount of entire illumination light emitted from the distal end of the optical fiber; and a control unit for controlling the amount of the illumination light emitted by the light source on the basis of the amount of emitted light converted by the conversion unit.

According to this aspect, when the distal end of the optical fiber is vibrated by the optical scanning section in the radial direction, the illumination light radiated on the subject from the distal end of the optical fiber is scanned on the subject, and return light generated at each irradiation position of the illumination light is detected by the return-light detection unit. By doing so, return light can be observed.

In this case, the amount of a portion of the illumination light emitted from the distal end of the optical fiber is detected by the emitted-light-amount detection unit, the amount of entire illumination light emitted from the distal end of the optical fiber is calculated by the conversion unit from the detected amount of light, and the amount of light emitted by the light source unit for supplying illumination light to the optical fiber is controlled by the control unit on the basis of the calculated amount of emitted light. By doing so, the amount of emitted light can be controlled to a desire amount, regardless of the difference between the amount of light emitted from light source unit and the amount of illumination light emitted from the optical fiber.

In the above-described aspect, a scope incorporating the optical fiber and the emitted-light-amount detection unit may be connected, such that the scope can be replaced, to a control device main body incorporating the control unit; the optical scanning endoscope device may include a memory unit that is provided in the scope and that stores a correlative relationship between the amount of light detected by the emitted-light-amount detection unit and the irradiation amount of the illumination light emitted from the scope; and the conversion unit may convert, into the irradiation amount of light, the amount of light detected by the emitted-light-amount detection unit on the basis of the correlative relationship stored in the memory unit, and the amount of the illumination light emitted by the light source may be controlled on the basis of the converted irradiation amount of light.

A portion of the illumination light emitted from the optical fiber is incident on the emitted-light-amount detection unit, and the remaining portion is emitted from the scope. The amount of illumination light incident on the emitted-light-amount detection unit is affected by a member in the scope. For this reason, the relationship between the amount of illumination light incident on the emitted-light-amount detection unit (i.e., the amount of light detected by the emitted-light-amount detection unit) and the irradiation amount of illumination light that is emitted from the scope and radiated on the subject differs for each scope. Given that, by providing, in the scope, the memory unit in which a pre-acquired correlative relationship between the amount of light incident on the emitted-light-amount detection unit and the irradiation amount of light is stored, it is possible to estimate the irradiation amount of light from the amount of light detected by the emitted-light-amount detection unit on the basis of the correlative relationship in the memory unit. Also, by controlling the light source on the basis of the obtained irradiation amount of light, the irradiation amount of illumination light radiated on the subject can be controlled to a desire amount.

In the above-described aspect, the memory unit may store the correlative relationship between the amount of light detected by the emitted-light-amount detection unit and the irradiation amount of the illumination light detected under a condition where the return light from the subject is not produced.

By doing so, a more accurate irradiation amount of illumination light radiated on the subject can be stored in the memory unit.

The above-described aspect may include a calculation unit for calculating an actual amount of the illumination light detected by the emitted-light-amount detection unit on the basis of the amount of light detected by the emitted-light-amount detection unit and the amount of return light detected by the return-light detection unit, wherein the conversion unit may convert, into the amount of entire emitted illumination light, the actual amount of light calculated by the calculation unit.

Return light may be mixed in the illumination light detected by the emitted-light-amount detection unit, and the amount of mixed return light correlates with the amount of return light detected by the return-light detection unit. Therefore, on the basis of the amount of return light detected by the return-light detection unit, the calculation unit can calculate an actual amount of the illumination light incident on the emitted-light-amount detection unit, excluding the amount of return light mixed in the illumination light. On the basis of the amount of emitted light converted from such an actual amount of light, even more accurate control is possible so that the amount of illumination light emitted from the optical fiber exhibits a desire amount.

In the above-described aspect, the control unit may control the amount of the illumination light emitted by the light source on the basis of the amount of emitted light converted from the amount of light that is detected by the emitted-light-amount detection unit when the distal end of the optical fiber is stationary or when the amplitude of vibration of the distal end is smaller than a prescribed threshold value.

By doing so, on the basis of the amount of light detected by the emitted-light-amount detection unit, even more accurate control is possible so that the amount of light emitted from the optical fiber exhibits a desire amount. When the amplitude of vibration of the distal end of the optical fiber is large, the amount of light detected by the emitted-light-amount detection unit greatly changes due to a large variation in the relative position between the emitted-light-amount detection unit and the distal end of the optical fiber, making it difficult to accurately detect the amount of entire emitted light.

The above-described aspect may include a light-blocking wall that is provided between the optical fiber and the emitted-light-amount detection unit and that restricts transmission of the illumination light, wherein the emitted-light-amount detection unit may detect the amount of illumination light passing through the light-blocking wall.

By doing so, because the light-blocking wall prevents light other than the illumination light emitted from the optical fiber from entering the emitted-light-amount detection unit, a more accurate amount of emitted illumination light can be obtained from the amount of light detected by the emitted-light-amount detection unit.

The present invention affords an advantage in that the amount of illumination light emitted from the optical fiber can be controlled to a desire amount.

### REFERENCE SIGNS LIST

100, 200, 300 Optical scanning endoscope device
1 Light source unit
2 Optical scanning section
3 Return-light detection unit
31 Return-light photodiode (return-light detection unit)
4 Emitted-light photodiode (emitted-light-amount detection unit)
6 Control unit
7R, 7G, 7B Laser light source (light source)
10 Illumination fiber (optical fiber)
14 Light-blocking wall
14a Window
15, 151 Memory unit
16 Calculation unit
19 Color filter

1. An optical scanning endoscope device comprising:
a light source configured to emit illumination light;
an optical fiber configured to guide the illumination light emitted from the light source to emit the illumination light from a distal end towards a subject;
an optical scanner configured to vibrate the distal end of the optical fiber in a radial direction of the optical fiber to scan the illumination light on the subject;
a return-light detector configured to detect return light that returns from the subject irradiated with the illumination light;
an emitted-light-amount detector configured to detect an amount of a portion of the illumination light emitted from the distal end of the optical fiber;
a converter configured to convert the amount of light detected by the emitted-light-amount detector into an amount of entire illumination light emitted from the distal end of the optical fiber;
a controller configured to control an amount of the illumination light emitted by the light source on a basis of the amount of emitted light converted by the converter; and
a calculator configured to calculate an actual amount of the illumination light detected by the emitted-light-amount detector on a basis of the amount of light detected by the emitted-light-amount detector and the amount of return light detected by the return-light detector,
wherein the converter is configured to convert, into the amount of entire emitted illumination light, the actual amount of light calculated by the calculator.

2. An optical scanning endoscope device comprising:
a light source configured to emit illumination light;
an optical fiber configured to guide the illumination light emitted from the light source to emit the illumination light from a distal end towards a subject;
an optical scanner configured to vibrate the distal end of the optical fiber in a radial direction of the optical fiber to scan the illumination light on the subject;
a return-light detector configured to detect return light that returns from the subject irradiated with the illumination light;
an emitted-light-amount detector configured to detect an amount of a portion of the illumination light emitted from the distal end of the optical fiber;
a converter configured to convert the amount of light detected by the emitted-light-amount detector into an amount of entire illumination light emitted from the distal end of the optical fiber; and
a controller configured to control the amount of the illumination light emitted by the light source on a basis of the amount of emitted light converted by the converter,
wherein the controller is configured to control the amount of the illumination light emitted by the light source on a basis of the amount of emitted light converted from the amount of light that is detected by the emitted-light-amount detector when the distal end of the optical fiber is stationary or when an amplitude of vibration of the distal end is smaller than a prescribed threshold value.

3. The optical scanning endoscope device according to claim 1,
wherein a scope incorporating the optical fiber and the emitted-light-amount detector is connected, such that the scope can be replaced, to a control device main body incorporating the controller,
the optical scanning endoscope device includes a memory that is provided in the scope and that stores a correlative relationship between the amount of light detected by the emitted-light-amount detector and an irradiation amount of the illumination light emitted from the scope, and
the converter is configured to convert, into the irradiation amount of light, the amount of light detected by the emitted-light-amount detector on a basis of the correlative relationship stored in the memory, and the amount of the illumination light emitted by the light source is controlled on a basis of the converted irradiation amount of light.

4. The optical scanning endoscope device according to claim 2,
wherein a scope incorporating the optical fiber and the emitted-light-amount detector is connected, such that the scope can be replaced, to a control device main body incorporating the controller,
the optical scanning endoscope device includes a memory that is provided in the scope and that stores a correlative relationship between the amount of light detected by the emitted-light-amount detector and an irradiation amount of the illumination light emitted from the scope, and
the converter is configured to convert, into the irradiation amount of light, the amount of light detected by the emitted-light-amount detector on a basis of the correlative relationship stored in the memory, and the amount of the illumination light emitted by the light source is controlled on a basis of the converted irradiation amount of light.

5. The optical scanning endoscope device according to claim 3, wherein the memory stores the correlative relationship between the amount of light detected by the emitted-light-amount detector and the irradiation amount of the illumination light detected under a condition where the return light from the subject is not produced.

6. The optical scanning endoscope device according to claim 4, wherein the memory stores the correlative relationship between the amount of light detected by the emitted-light-amount detector and the irradiation amount of the illumination light detected under a condition where the return light from the subject is not produced.

7. The optical scanning endoscope device according to claim 2, further comprising:
a calculator configured to calculate an actual amount of the illumination light detected by the emitted-light-amount detector on a basis of the amount of light detected by the emitted-light-amount detector and the amount of return light detected by the return-light detector,
wherein the converter is configured to convert, into the amount of entire emitted illumination light, the actual amount of light calculated by the calculator.

8. The optical scanning endoscope device according to claim 1, wherein the controller is configured to control the amount of the illumination light emitted by the light source on a basis of the amount of emitted light converted from the amount of light that is detected by the emitted-light-amount detector when the distal end of the optical fiber is stationary or when an amplitude of vibration of the distal end is smaller than a prescribed threshold value.

9. The optical scanning endoscope device according to claim 1, further comprising:
a light-blocking wall configured to restrict transmission of the illumination light on an outward side in the radial direction of the optical fiber,
wherein the emitted-light-amount detector is disposed on a side more outward than the light-blocking wall in the radial direction,
the light-blocking wall has a window for transmitting the illumination light at a position facing the distal end of the optical fiber in the radial direction, and
the emitted-light-amount detector is configured to detect the amount of illumination light passing through the window.

10. The optical scanning endoscope device according to claim 2, further comprising:
a light-blocking wall configured to restrict transmission of the illumination light on an outward side in the radial direction of the optical fiber,
wherein the emitted-light-amount detector is disposed on a side more outward than the light-blocking wall in the radial direction,
the light-blocking wall has a window for transmitting the illumination light at a position facing the distal end of the optical fiber in the radial direction, and
the emitted-light-amount detector is configured to detect the amount of illumination light passing through the window.
